(19) United States
(12) Patent Application Publication
(10) Pub. No.: US 2016/0287749 A1
(43) Pub. Date: Oct. 6, 2016

(54) RECONSTITUTED AMNIOTIC MEMBRANE-AMNIOTIC FLUID COMBINATION TISSUE GRAFT

(71) Applicant: Applied Biologics, LLC, Scottsdale, AZ (US)

(72) Inventor: Edward Britt, Scottsdale, AZ (US)

(21) Appl. No.: 14/986,167

(22) Filed: Dec. 31, 2015

Related U.S. Application Data
(60) Provisional application No. 62/099,007, filed on Dec. 31, 2014.

(51) Int. Cl. A61L 27/36

(52) U.S. Cl. CPC A61L 27/3633; A61L 2300/00

(57) ABSTRACT

The invention relates to preparations and methods of creating preparations of reconstituted amniotic membrane utilizing amniotic fluid, for use as combination tissue grafts in surgical and minimally invasive medical therapy of injury and disease. The preparations maximize available quantities of viable mesenchymal stem cells and non-cellular bioactive compounds to enhance therapeutic efficacy. The tissue graft preparations are liquids and/or semi-viscous fluids which may be intraoperatively transplanted at the recipient site using a needless syringe or by non-operative percutaneous injection through a hypodermic needle.

### CROSS REFERENCE TO RELATED APPLICATIONS

This application claims the benefit of U.S. Provisional Application No. 62/099,007, filed Dec. 31, 2014, the contents of which are incorporated entirely herein by reference.

### BACKGROUND OF THE INVENTION

1. Technical Field

This invention relates to the preparation of a combination tissue graft using amnion and amniotic fluid; in particular, the invention relates to the preparation and use of a mammalian combination tissue graft formed by reconstituting dried, ground amniotic membrane with amniotic fluid.

2. State of the Art

Amniotic membrane, specifically human amniotic membrane, has been used in surgery for over one hundred years. The amnion interstitial matrix contains a complex biologic soup of growth factors, inflammatory mediators, immuno-modulators, and other active biomolecules. Additionally, amniotic membrane is rich in embryonic stem cells.

Amniotic membrane is used in a variety of surgical procedures as an adjunct to healing, and to minimize formation of scar tissue and adhesions. The amniotic membrane is typically dried prior to packaging, sterilization, and storage. Some preparations, however, reconstitute the dried amniotic membrane using a tissue preservative solution prior to the packaging and sterilization for storage. The medium used to reconstitute the dried amniotic membrane is typically an isotonic solution containing water and electrolytes, but no growth factors, other active biomolecules, or additional extraembryonic stem cells.

Accordingly, what is needed is a preparation formed from dried amniotic membrane reconstituted in a fluid which supplants the tissue proliferative, antimicrobial, immuno-modulatory, and anti-inflammatory properties of amniotic membrane.

Citation of documents herein is not an admission by the applicant that any is pertinent prior art. Stated dates or representation of the contents of any document is based on the information available to the applicant and does not constitute any admission of the correctness of the dates or contents of any document.

### DISCLOSURE OF EMBODIMENTS OF THE INVENTION

Disclosed is a reconstituted combination tissue graft comprising a dried amniotic membrane and an amniotic fluid, wherein the amniotic fluid rehydrates the dried amniotic membrane.

In some embodiments, the dried amniotic membrane is morcellized. In some embodiments, the dried amniotic membrane is ground.

In some embodiments, the combination tissue graft further comprises a non-amniotic fluid liquid. In some embodiments, the non-amniotic fluid liquid is an isotonic electrolyte solution. In some embodiments, the non-amniotic fluid liquid is a cryoprotectant. In some embodiments, the non-amniotic fluid liquid comprises an isotonic electrolyte solution and a cryoprotectant.

In some embodiments, the amniotic membrane and amniotic fluid are mammalian. In some embodiments, the amniotic membrane and amniotic fluid are from one individual donor. In some embodiments, the amniotic membrane and amniotic fluid are from more than one individual donor. In some embodiments, the combination tissue graft is lyophilized. In some embodiments, the combination tissue graft is a fluid. In some embodiments, the combination tissue graft is a semi-solid gel.

Disclosed is a reconstituted combination tissue graft comprising a dried amniotic membrane; a processed amniotic fluid derivative comprising a protein released from a disrupted cell; and a hydrating fluid, wherein the hydrating fluid rehydrates the dried amniotic membrane.

In some embodiments, the protein is a growth factor. In some embodiments, the disrupted cell is an amniocyte. In some embodiments, the hydrating fluid is an amniotic fluid.

Disclosed is a method of forming a combination tissue graft comprising the steps of grinding an amnion; and mixing the ground amnion with a quantity of processed amniotic fluid derivative to form a combination tissue graft.

In some embodiments, the method further comprises centrifuging the quantity of amniotic fluid prior to mixing; decanting a supernatant; suspending a centrifuge pellet in a suitable fluid; and repeating the centrifuging, decanting, and suspending steps to form a processed amniotic fluid derivative.

In some embodiments, the method further comprises a step lyophilizing the combination tissue graft.

The foregoing and other features and advantages of the present invention will be apparent from the following more detailed description of the particular embodiments of the invention, as illustrated in the accompanying drawings.

### BRIEF DESCRIPTION OF THE DRAWINGS

FIG. 1 is a representation of a reconstituted amniotic membrane-amniotic fluid combination tissue graft.

FIG. 2 is a flow chart outlining steps of a method 100 of forming a combination tissue graft.

FIG. 3 is a flowchart diagraming steps of a method 200 of forming a combination tissue graft.

FIG. 4 is a flowchart diagraming steps of a method 300 of forming a combination tissue graft.

FIG. 5 is a flowchart diagraming steps of a method 500 of forming a combination tissue graft.

### DETAILED DESCRIPTION OF EMBODIMENTS OF THE INVENTION

Fetal placental membranes (“PMs”) occupy a unique position in the field of regenerative medicine. This tissue, which derives solely from the developing embryo and fetus, comprises amnion (amniotic membrane or “AM”) and chorion (chorionic membrane or “CM”) fused at a basement-membrane/stroma interface and contains a dense concentration of extraembryonic mesenchymal stem cells (“SCs”) in an interstitial matrix rich with multiple classes of biologically active molecules.

The AM is a single layer of epithelial cells—amniocytes—on a thick basement membrane/connective tissue stroma. It derives from the embryonic epiblast, which is adjacent to the primitive streak and contiguous with cells giving rise to the notochord, and grows into a fluid-filled sac enveloping the developing fetus.

The CM is a more complex tissue, adjacent to and invading the maternal uterine wall, but arising from the embryonic trophoblast. In contrast to the histologically simple amniotic membrane, the chorion is more complex. The trophoblast is a tissue on the uterine surface of the chorion and contains subpopulations of cells. One cell population, the extravillous cytotrophoblast, invades the maternal endometrium. Another, the syncytiotrophoblast, forms a syncytium of densely nucleated cytoplasm covering the chorionic villi and directly contacting the maternal blood. Like the AM, the CM is also rich in undifferentiated extraembryonic mesenchymal stem cells. Unlike the AM, CM is used less extensively as a tissue graft because of its immunogenicity. This arises from residual bits of decidua (maternal endometrial tissue contacting the placenta). Additionally, and perhaps more importantly, CM tissue components of fetal origin, including fetal blood vessels, connective tissue, endothelial cells, and residual fetal blood elements, elicit an immunological response in the tissue graft recipient leading to rejection of the tissue graft. And although the CM stromal layer, which is adjacent to the basement membrane of the AM, contains non-immunogenic SC's and large/small biomolecules, the trophoblast and fetal connective tissue components express HLA Class I and HLA-D cell surface antigens which allow development of a full host immune response to grafted CM. Consequently, intact AM which is manually “peeled” from the AM at the stromal interface is used in various tissue graft preparations whereas use of CM is limited by its antigenicity. The CM is a source of beneficial tissue, SCs, and biomolecules. When the placental membranes are received from a volunteer donor and the CM is discarded, however, at least half of the donor's PM SCs and bioactive molecules are lost.

Amniotic fluid (“AF”) is an additional source of beneficial material. AF, which bathes the fetus and is contained by the AM, is a biologically complex substance which, although extensively studied, remains incompletely defined and understood. It is known that AF contains large numbers of suspended amniocytes, SCs, and non-cellular components including small molecules, growth factors, hormones, immunomodulators, and antimicrobials. Small molecules in solution within the AF include electrolytes, glutamine (important for nucleic acid synthesis), arginine (necessary for placental angiogenesis), and hyaluronic acid (inhibits collagen synthesis; may mitigate scaring and fibrosis during wound healing). Growth factors identified in AF include transforming growth factor alpha (“TGF-α”), epidermal growth factor (“EGF”), insulin-like growth factor I (“IGF-1”), hyaluronic acid-stimulating factor, macrophage colony-stimulating factor (“M-CSF”), and granulocyte colony-stimulating factor (“G-CSF”). These growth factors all potently stimulate proliferation of stem cell and many non-progenitor cell-types in both fetal and adult cells and tissues. Hormones identified in AF include erythropoietin (“EPO”), which promotes proliferation of red blood cell progenitors and may stimulate growth of the gut endothelium. Immunomodulators and antimicrobials in AF include α-defensins, lactoferrin, lysozyme, bactericidal/permeability-increasing protein, calprotectin, secretory leukocyte protease inhibitor, psoriasin, a cathelizidin, and various polyamines with antimicrobial properties. Additionally, cellular immune components present in AF include monocytes, macrophages, and histiocytes. In addition to all of these substances, AF almost certainly contains additional compounds which also provide benefits to a tissue graft recipient.

Although AF contains many phenotypically distinct subpopulations of SCs, these cells generally do not express HLA Class I, II, and other cell surface antigens in a manner sufficient to elicit a host immune response, as measure by a mixed lymphocyte reaction (“MLR”). Therefore, AF is a valuable source of biologically active molecules and immune-privileged pluripotent SCs.

Collection of AF for preparation of tissue grafts during the peri-partum period in the time prior to a vaginal delivery is not possible. Amniocentesis under sterile, controlled conditions prior to the peri-partum period is source of sterile AF. Amniocentesis, however, when performed to obtain a tissue donation should not justify even a small risk to the developing fetus. Amniocentesis carries a risk of spontaneous abortion of up to 0.5% when electively performed in the second trimester. Consequently, AF during pregnancy cannot, safely and practically, be collected in significant bulk from a pool of volunteer donors prior to or during a vaginal delivery.

Amniotic fluid may be collected under sterile conditions in the operating room during an elective Cesarean section delivery with essentially no risk to the infant or the mother. There are just under 4 million births per year in the United States of which approximately 33%—1.32 million overall—are by Cesarean delivery. Fetal placental membranes, including AM and CM, however, may additionally be collected during a routine vaginal delivery. The bacterial contamination that occurs with vaginal delivery of the placenta is minimal in an uncomplicated delivery and may be addressed. Fetal membranes use as tissue grafts collected from a vaginally delivered placenta may be effectively treated with sterile washings using topical antibiotic and non-tissue-toxic antimicrobial solutions immediately following delivery and thereafter. Therefore, AM but not AF is potentially available for use as a combination tissue graft from between 3.5 and 4.0 million births annually in the U.S.

Conversely, AM suitable for use in a combination tissue graft is not universally available through a Cesarean delivery where suitable AF is obtained. Gross contamination rendering the AM unsuitable for grafting may occur during the delivery itself, or later during processing and/or packaging.

As briefly mentioned, AM may be collected from suitable volunteer donors and processed for storage prior to use as a tissue combination tissue graft in a variety of surgical procedures. AM is used in a plethora of surgical procedures and non-surgical applications. Some examples include use of AM as a biologic dressing, an adjunct to healing of surgically repaired bone, tendon, other soft tissue, and open wounds; a means to militate the formation of scar tissue and adhesions, and other beneficial applications in surgery and non-surgical minimally invasive medical therapies. AM and AM derivatives are used as biologic dressings containing a source of SCs and growth factors to treat burns, skin pressure ulcers, other chronic open wounds, corneal ulcers, and as a dressing following corneal transplant and other ocular procedures. AM tissue combination tissue grafts are used to address soft tissue defects and facilitate healing following debridement and repair of damaged cartilage, tendon, bone, and muscle tissue. AM is under investigation as a connective tissue scaffolding for tissue and organogenesis using extraembryonic SCs and other progenitor cells.

In all of these and other applications, there is strong evidence that the presence of viable SCs and active biomolecules in the AM-derived dressing or combination tissue graft improves healing across a broad range of tissue types, locations within the body, and applications. Reporting of clinical results may eventually lead to the use of AM and AM-derived preparations as a standard therapy and possibly even a best practice for the treatment of a variety of conditions. Such reporting requires continued laboratory experimentation and human clinical trials to generate additional data for review and interpretation in light of currently available practices and results therefrom. Meaningful interpretation of these data however, depends on reproducibility. Reproducibility requires standardization of materials and techniques. Such standardization in this area should include the delivered dose of SCs, total tissue weight per volume, and the concentration of small and large-molecule biologically active compounds present in the combination tissue graft used.

Preparation and sterilization of AM for later use as a tissue combination graft typically includes drying, packaging, sterilization, and storage. Drying discourages bacterial growth and helps maintain sterility during storage. Drying, however, has negative effects on AM. Drying may be accomplished by heating or freezing in a partial vacuum (lyophilization or “freeze drying”) to minimize water-ice crystal formation and cellular disruption. Although some viable SCs are preserved by drying under controlled conditions (use or a suitable cryoprotectant combined with controlled-rate freezing) other SC's die during processing. It is not fully known how drying and storage affect the concentration of the biologically active non-cellular components of AM, though a significant decrease in concentration of intact proteins and other large biomolecules is possible. Sterilization by heat or radiation destroys the cellular components of AM preparations, including SCs. Thermal or irradiative sterilization methods may also denature proteins and alter or destroy other large biologically active molecules.

Some tissue graft preparations reconstitute the dried AM using a tissue preservative solution prior to packaging and storage. The medium used to reconstitute the dried amniotic membrane is typically a buffered isotonic solution containing water and electrolytes, but no growth factors, other active biomolecules, or additional SCs.

What is lacking in the prior art, therefore, is an AM-derived combination tissue graft preparation incorporating an effective concentration of SCs and active biomolecules while minimizing loss of SCs and non-cellular tissue elements available from an individual donor or largest possible pool of volunteer donors.

Embodiments of this invention address these fundamental AM combination tissue graft requirements—high concentration of viable SCs and beneficial biomolecules in a standardized preparation with no antigenic material and minimal waste of available donor tissue—by forming a combined tissue graft comprising a preparation of dried particulate AM rehydrated by a reconstituted AF-derived suspension and frozen under controlled conditions to preserve SC viability.

Disclosed is a combination tissue graft preparation, including a method of forming same, comprising dried and ground amniotic membrane reconstituted with amniotic fluid. In some embodiments, the preparation further comprises a standardized quantity of viable SCs per unit volume, a standardized weight of ground AM per unit volume, or both. The preparation is used by medical providers as a combination tissue graft, either by intraoperative application or injection, non-operative percutaneous injection, or direct application to injured, ischemic, infected, or otherwise damaged tissue. The preparation is also used by laboratory researchers as a reproducible source of standardized material for basic science research of the effects of combination AM/AF preparations on healthy, diseased, and damaged tissue; in the field of regenerative medicine; and in other scientific disciplines. The use of ground, dried AM reconstituted in AF, with or without additional hydrating fluids including but not limited to isotonically balanced electrolyte solutions and/or cryoprotectant, maximizes delivery of SCs and a wide range of beneficial biologic substances within a non-antigenic liquid combination tissue graft to the treatment site.

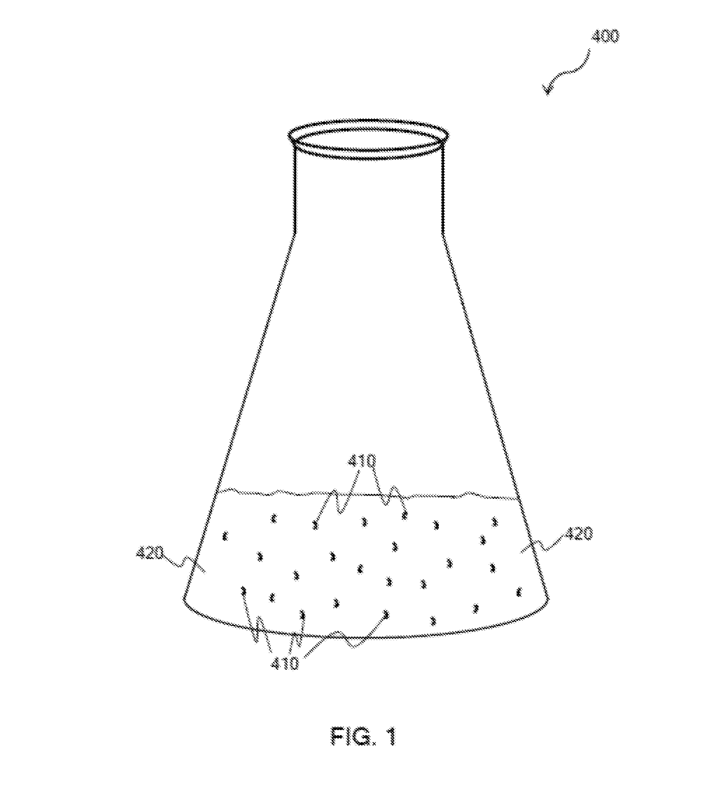

FIG. 1 show a representation of a reconstituted combination amniotic membrane-amniotic fluid tissue graft 400. Details regarding the composition and preparation of combination tissue graft 400 are provided herein below and throughout this disclosure. Combination tissue graft 400 comprises fragments of a dried amniotic membrane 410 reconstituted with a processed amniotic fluid derivative 400. As will be discussed herein below, amniotic fluid derivative 420 rehydrates the dried or partially dried fragments of amniotic membrane 410. In some embodiments, amniotic fluid derivative 420 is fresh amniotic fluid without any processing or addition of other material. In some embodiments, amniotic fluid derivative 420 is a processed amniotic fluid derivative which has been reconstituted with a suitable fluid following serial washings discussed in detail herein below. FIG. 1 shows combination tissue graft 400 contained in a flask. This is only for illustration purposes. Combination tissue graft, in some embodiments, is contained in a variety of packaging means, including in sealed single-dose vials, hypodermic syringes, multi-dosed vials, and as a frozen, lyophilized material which is reconstituted by the addition of a suitable fluid when ready for use, such as a buffered isotonic electrolyte solution, for example.

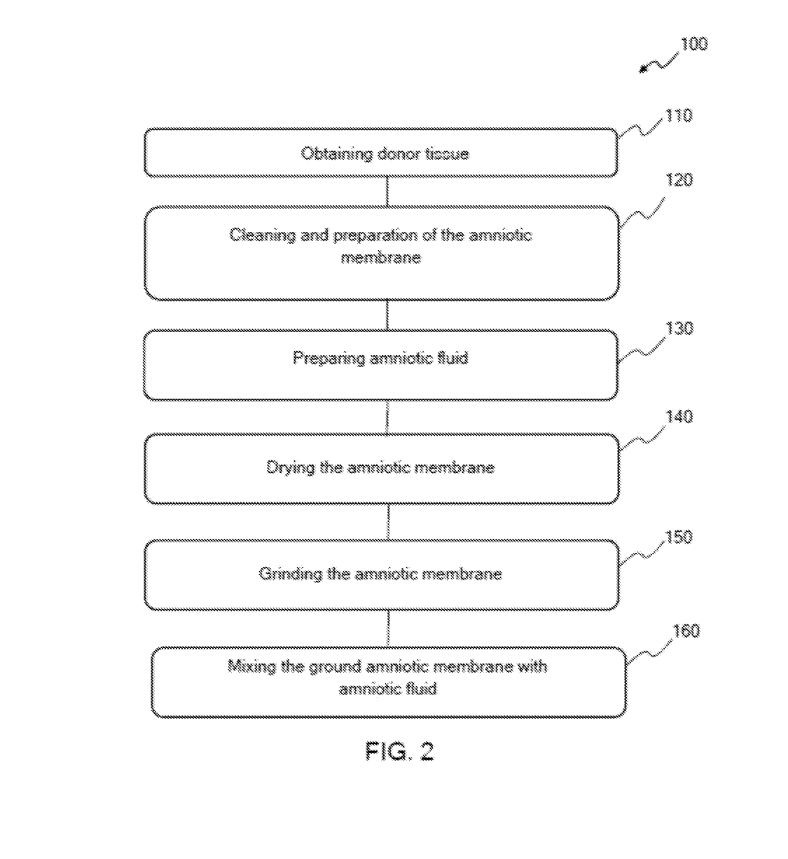

FIG. 2 shows an overview 100 of the processing steps utilized through some embodiments of the invention to create combination tissue graft 400. Overview 100 requires an amnion. In some embodiments of the invention, the AM comes from a volunteer human donor. Accepting amniotic tissue from volunteer donors and excluding non-volunteer and/or paid donors from the donor pool is consistent with internationally well-established tissue donation protocols because it reduces the chance that an infectious agent present in the donor will be transmitted to the graft recipient, resulting in an infection in the recipient. Screening of potential volunteer donors, therefore, includes obtaining a comprehensive past medical and social history, complete blood count, liver and metabolic profile, and serologic testing for HBV, HCV, and HIV, in some embodiments.

In some embodiments, donor tissue is obtained during delivery by elective Cesarean section. In some example embodiments, intraoperative aspiration of AF is performed immediately prior to delivery and the aspirated AF is sealed in a plastic specimen container. Following Cesarean delivery of the infant, the placenta is delivered. The combined fetal membranes (AM and CM) are dissected from the maternal placental plate (decidua). The combined fetal membranes are then placed in a second sterile specimen container and a quantity of 0.9% sterile saline is added sufficient to completely submerge the combined fetal membranes. The individual sterile containers containing the feta placental membranes and amniotic fluid collected under sterile conditions in the operating room are then placed together in a donor tissue specimen bag. This bag is placed within a second sterile bag, sealed, and taken from the operating room for packaging in an insulated ice-bath container. The container is then immediately transported to the processing facility by staff who rotate on call, such that there is minimal delay following delivery before the donor tissue arrives at the separate facility for processing.

Despite the preference for a Cesarean-delivered AM in order to increase the pool of potential donors and other of the aforementioned reasons, vaginally delivered fetal membranes are utilized in some embodiments. Great care must be afforded the vaginally-delivered placental tissue to prevent microbial contamination. Vaginally-delivered fetal membranes are not acceptable donor tissue if there is fecal or other grossly visible contamination, or if there is contact of the placental membranes with clothing, bedding, non-sterile unprepped skin, or other non-sterile surfaces during delivery or prior to sterile packaging. Neither a vaginally-delivered AM nor a Cesarean-delivered AM is acceptable donor tissue if there is visible staining of the fetal membranes with meconium. Following delivery, the steps for preparing vaginally delivered fetal membranes are the same as the above description of preparing Cesarean-delivered fetal membranes. A fully gowned-and-gloved staff member processes the fetal membranes on a sterile field established on a back table, or similar surface, in the labor/delivery room. An additional step comprising rinsing the vaginally delivered fetal membranes with an antimicrobial solution is used in some embodiments. After washing with 0.9% sterile saline, the vaginally delivered dissected fetal membranes are washed with a topical antimicrobial solution. Examples of the topical antimicrobial solution used to wash the vaginally delivered fetal membranes, in some embodiments, are a 0.5% aqueous solution of glutaraldehyde (which is then washed off the donor tissue using a final rinse of 0.9% sterile saline prior to packaging), a Penicillin-Streptomycin solution comprising 50-100 International Units (“IU”) per ml of penicillin and 50-100 micrograms/ml of Streptomycin, or a 0.0125% aqueous solution of sodium hypochlorite. These examples are not meant to be limiting. Other antimicrobial solutions toxic to infectious microorganisms at non-cytotoxic concentrations may also be used. The fetal membranes, following the antimicrobial washing, are then placed in a sterile specimen container, covered with 0.9% sterile saline solution, and sealed in sequential sterile bags as described above for Cesarean-delivered fetal membranes. The prepared, sealed, labeled, recorded, and packaged donor fetal membranes are then delivered to the separate tissue processing facility, as described above.

Immediately upon receipt at the processing facility, the shipping label is examined and information regarding the specimen and donor is recorded. The shipping container is examined for integrity, including confirmation of an intact tamper-proof seal. The shipping container is then opened and the inner bag containing the placental membranes and amniotic fluid is examined. An infrared temperature sensor is directed at the tissue bag to confirm a temperature of between 6 and 10 degrees Celsius. If there is any indication of damage to the outer container, the inner bag containing the placental membranes and amniotic fluid is examined with particular care. If damage to the inner bag is identified or the tamper-proof seal is broken or damaged, the specimen is not used to prepare the tissue graft. A donor/specimen data sheet within the container is then reviewed to validate the donor's credentials. The information on the data sheet is compared to the donor ID on the specimen bag to confirm the data sheet for the donor matches the specimen. This information is recorded and included in the permanent batch record for that specific donor. These credentials include donor lot numbers and expiration dates. All validation dates and times are confirmed. A donor tissue specimen that is unacceptable for any reason is discarded. The date, time, and hospital from which the donor specimen was received is recorded. The outside of the bag containing the two separate sterile specimen containers is then sprayed with isopropyl alcohol and manually wiped down. The logged and cleaned specimen bag containing the donor placental membranes and amniotic fluid is then stored in a locked refrigerator in an ice water bath, but not frozen.

Following first step 110 and receipt of the donor tissue, second step 120, comprises cleaning and preparation of the amniotic membrane for grinding or morcellizing, as practiced in some embodiments shown in FIG. 2. Under strict sterile technique, the specimen bag is opened using sterile scissors and the donor specimen comprising placental membranes and amniotic fluid is carefully poured into a large sterile basin. Using sterile forceps, the AM is peeled from the CM, which separates at the AM basement membrane/CM stromal interface. The AM is placed on a sterile cutting board, CM-side facing up. The CM side is gently wiped with sterile cloth towels, taking care to remove any adherent bits of CM and clotted blood which may not have been completely rinsed from the AM immediately following the delivery prior to packaging. Both sides of the AM are once again washed with sterile 0.9% saline and rinsed with an antimicrobial solution in some embodiments, such as 0.5% aqueous solution of glutaraldehyde for example.

FIG. 2 also shows step 130, preparation of amniotic fluid. In some embodiments, step 130 comprises removing AF from the large sterile basin in 50 ml aliquots using a sterile pipette. Each aliquot of AF is pipetted into a centrifuge tube, which is capped and then spun-down into a pellet and supernatant by centrifuging at 1,200 RPM for 10 minutes. The supernatant, containing water, electrolytes, and other solutes, is pipetted from the tube and discarded. The pellet, containing SCs, proteins, and other large molecules with associated insoluble compounds is reconstituted in a suitable fluid. In some embodiments, the suitable fluid is a buffered isotonic electrolyte solution. In some embodiments, the suitable fluid is a cryoprotectant. In some embodiments, the suitable fluid is a sterile, non-pyrogenic isotonic solution of sodium chloride, sodium gluconate, sodium acetate, potassium chloride, and magnesium chloride buffered to a pH of 7.4 with sodium hydroxide (i.e., Plasma-Lyte A™). In some embodiments, the suitable fluid is a 10% solution of dimethylsulfoxide (“DMSO”). In some embodiments, the suitable fluid is a 50% solution of glycerol. A quantity of this solution just adequate to suspend the pellet material for removal by pipette is used, usually one ml or less. The washed, re-constituted AF material from two tubes in the first spin-down is combined into one centrifuge tube and centrifuged for a second time at 1,200 rpm for 10 minutes. Again, the supernatant is pipetted off the solid pellet at the bottom of the tube, which is again re-constituted in a fresh quantity of suitable fluid, pipetted from the tube, and combined with a second washed re-constituted specimen. This sequence is repeated a third time, after which the centrifuge tube contained the thrice-washed pellet and supernatant is re-constituted in a quantity (1.0 ml to 5.0 ml, for example; not meant to be limiting) of fresh suitable fluid and the centrifuge tubes are temporarily stored in a water-ice bath. Prior to storage, a 0.5 ml sample from each lot of washed, re-constituted processed AF derivative is removed for a cell count. The number if intake SCs/cc of suspension is calculated and recorded for later standardization of SC concentration per unit volume of the final combination tissue graft preparation.

In some embodiments, the sealed, sterile plastic specimen container with AF is refrigerated but not frozen, and is not centrifuged and washed as described above. In some embodiments, the AF is combined with a cryoprotectant, such as DMSO at a 5% concentration by weight, frozen at a controlled rate to −80° C., and stored for later thawing and use in forming a combined tissue graft. The use of DMSO is not meant to be limiting. Other suitable cryoprotectants, such as a solution of 50% glycerol for example, may be used.

Step 140, also shown in FIG. 2, comprises drying of the AM as practiced in some embodiments of the invention. Using sterile scissors, the cleaned and treated AM from step 120 is cut into pieces measuring approximately 2×2 centimeters (“cm”) or approximately 4×4 cm and placed in a sterile pan for drying. In some embodiments, drying takes place at ambient conditions of temperature and humidity. In some embodiments, drying is performed in a drying oven under controlled temperature for a controlled time.

Step 150 of overview 100 shown in FIG. 2, in some embodiments, comprises grinding the AM. In some embodiments, the now dried AM is then removed from the drying rack and placed in a temperature-controlled ball-grinding mill (i.e. “CryoMill” for cryogenic grinding, manufactured by Retsch Corporation, Haan, Germany). The grinding jar and balls for the mill are weighed prior to placement of a quantity of dried AM in the grinding jar. After placement in the grinding jar, the dried AM is pre-cooled to minus 196° Celsius and then ground for approximately 4 minutes. This process results in an AM particle size of 5 microns. In some embodiments, grinding proceeds for longer than 4 minutes, resulting in smaller particle size. In some embodiments, grinding proceeds for less than four minutes, resulting in larger particle size. The grinding jar is again weighed, and the weight of milled AM contained within is determined. In some embodiments, fresh AM us placed in the grinding jar for freezing and grinding without first drying the AM.

In some embodiments, the AM is morcellized but not ground. A morcellized amnion may comprise non-viable amniocytes which are disrupted along with non-disrupted, viable amniocytes. Preparations with disrupted amniocytes have a higher concentration of growth factors, other functional protein and peptide molecules, and other biologically active molecules which are released into the preparation from the disrupted cells. Non-disrupted viable amniocytes comprise SCs which become engrafted into host tissue and participate in tissue regeneration and lend other highly beneficial, therapeutic effects to the tissue graft.

In these and similar embodiments utilizing morcellized AM, under sterile conditions, AM is cut into approximately 1 cm-wide strips using tissue scissors. The cut strips of AM are then morcellized using a variable speed tissue homogenizer at between 500 and 1000 rpm for a limited time. Morcellization is stopped when the AM is grossly shredded into tiny pieces by visual inspection, typically after five to fifteen seconds. The individual pieces are visible and fall within in an approximate range of 0.1 mm to 1.0 mm in size. The morcellized AM is then dried. In some embodiments, the morcellized AM is dried in a sterile container within a drying oven at controlled temperature for a set time. In some embodiments, the morcellized AM is dried in a sterile container under ambient conditions.

In some embodiments, AM which has been previously processed, such as in a dried, partially dried, or fresh state; packaged; and sterilized by irradiation is used for grinding or morcellization.

Step 160 of overview 100 shown in FIG. 2, in some embodiments, comprises mixing ground or morcellized AM with AF, centrifuged and decanted AF, or processed AF derivative to form the combination tissue graft. In some embodiments, ground AM is hydrated with AF, centrifuged and decanted AF, or processed AF derivative, with or without a second suitable fluid, by mixing step 160.

Prior to mixing step 160, the weighed milling jar containing the dried, ground AM is opened and the milled AM is washed from the jar and balls using a quantity of suitable fluid, approximately 50 ml of sterile isotonic saline solution, for example. Just enough solution is added to liquefy and partially reconstitute the milled AM. The exact quantity is recorded so that in addition to a standard cell count (based upon the initial donor cell count of SCs/ml of the re-constituted processed AF derivative or other hydrating fluid described in step 130 above), a standardized concentration by weight of AM per unit volume of re-constituted AF is also provided for the completed tissue graft, in some embodiments of the invention. The reconstituted AM in reconstituted AF (“AMFL”) therefore, has a known weight of AM per volume of AMFL. In some embodiments, the concentration by weight of AM per unit volume of tissue graft is chosen to form a combination tissue graft of a desired viscosity. In some embodiments, a suitable gelling agent, such as a prepared collagen gel for example, is added to the combination tissue graft to create a high-viscosity fluid or gel consistency.

In some embodiments, step 160 comprises reconstitution and combination of the ground AM with fresh, unmodified AF. In some embodiments, centrifuged, unwashed AF from which a portion of the supernatant has been decanted is used, such that the volume of centrifuged, decanted AF is the volume necessary to create a desired standardized concentration by weight of ground AM in the formed combination tissue graft product. In some embodiments, cryopreserved AF is used. In some embodiments, the AF or AF preparation is from the same donor as the AM. In some embodiments, the AF or AF preparation is from a different donor as the AM. In some embodiments, the AF, centrifuged decanted AF, or processed AF derivative is from pooled multiple donors. In some embodiments, the ground AM is from pooled multiple donors. In some embodiments, the AF is from a non-human mammalian species. In some embodiments, the AM is from a non-human mammalian species.

Step 160 is completed by combining quantities of AMFL, washed and re-constituted AM, a cryoprotectant, and buffered isotonic solution to form the completed combination tissue graft 400. Sterile materials are used and sterile technique is maintained. In some embodiments, a previously recorded weight per volume of AM and SC per volume of AF are noted such that the completed tissue graft is a known, standardized, reproducible product. An example buffered isotonic solution used in some embodiments to create the AMFL and reconstituted AF is Plasma-Lyte A (manufactured by Baxter International, Inc., Deerfield, Ill.). An example of a cryoprotectant used in some embodiments is CryoStor CS-10, a 10% solution of dimethylsulfoxide (“DMSO”) (manufactured by BioLife Solutions, Inc., Bothel, Wash.). These examples are not meant to be limiting; similar products may be compounded or obtained from other manufacturers for use in preparation of the tissue graft. Standardized dilution tables are pre-calculated based upon the initial donor cell count completed previously in step 130. Once the final dilution ratios have been confirmed and prepared, the measured individual components are poured into a large beaker and gently suspended by gently swirling the beaker and/or stirring with a glass rod or other suitable instrument.

In some embodiments, a small quantity of combination tissue graft 400, approximately 0.5 cc's for example, is drawn into a sterile 2 cc syringe and extruded through a 25 gauge needle to ensure that combination tissue graft 400 is sufficiently fluid to be percutaneously or intraoperatively injected into the recipient tissue bed. In some embodiments, the viscosity of the combination tissue graft 400 is further adjusted by mixing an additional measured quantity of buffered isotonic solution with the combination tissue graft 400, and recording the final concentration of AM and SC per ml accordingly. In some embodiments, the final concentration of AM and/or SC per ml is adjusted with additional buffered isotonic solution to an end-user's pre-ordered concentration requirement, based upon the intended use of combination tissue graft 400.

In some embodiments, step 160 also comprises determining the quantity of combination tissue graft 400 requested by end user based upon the intended use of the combination tissue graft 400. In some embodiments, combination tissue graft 400 is packaged in standard SC concentrations, AM concentrations, and total volumes. In some embodiments, combination tissue graft 400 is packaged in standard differing viscosities based upon the mode used for delivery (injection versus intraoperative application, for example) and intended therapeutic use.

In some embodiments, combination tissue graft 400 is then pipetted into empty product vials and placed in a lyophilization unit for controlled removal of water and other volatiles prior to final packaging and shipping. Lyophilization methods are well known in the art, and any suitable unit and/or lyophilization protocol may be used to lyophilize the packaged tissue graft 400. The packaging vials of lyophilized tissue graft are then sterilely sealed, labeled, and cooled in a controlled-rate freezer to minus 80° Celsius.

In some embodiments, combination tissue graft 400 containing a cryoprotectant is frozen in a controlled-rate freezer without lyophilization prior to freezing.

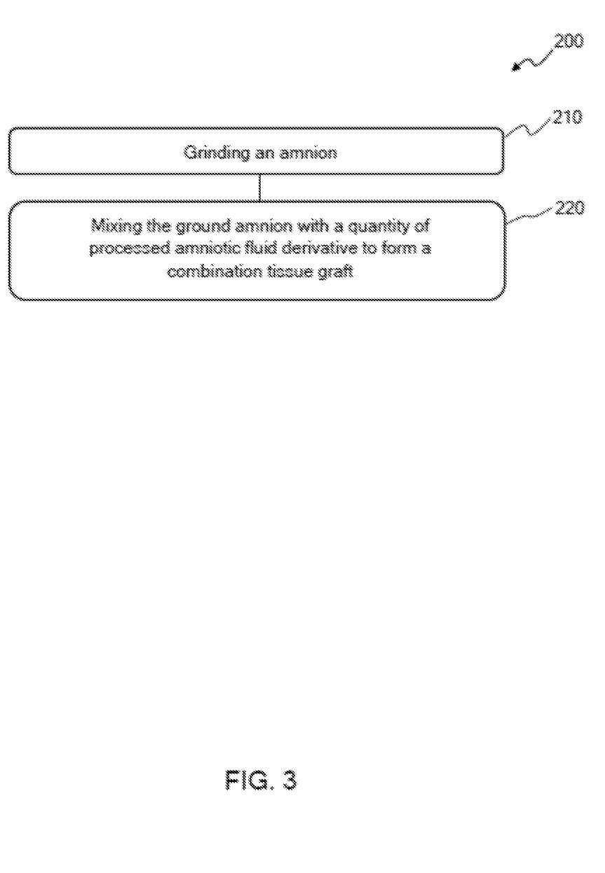

FIG. 3 shows a method 200 of forming a reconstituted amniotic membrane-amniotic fluid combination tissue graft 400. Step 210 of method 200 shown in FIG. 3 comprises grinding an amnion. In some embodiments, grinding is performed using a cryomill as discloses herein above. In some embodiments, fresh, non-dried AM is frozen in a cryomill immediately prior to grinding. In some embodiments, a dried or partially dried AM is frozen in the cryomill immediately prior to grinding. Specific details of various means used to perform step 210 of method 200 in some embodiments of the invention have been discussed herein above.

FIG. 3 also shows step 220 of method 200 comprising mixing the ground amnion with a quantity of processed amniotic fluid derivative to form a combination tissue graft. In some embodiments, processed amniotic fluid derivative is fresh amniotic fluid collected under sterile conditions and refrigerated without freezing, without further processing or addition of other material. In some embodiments, processed amniotic fluid derivative is a processed, concentrated amniotic fluid reconstituted with a suitable fluid following serial washings as discussed herein above.

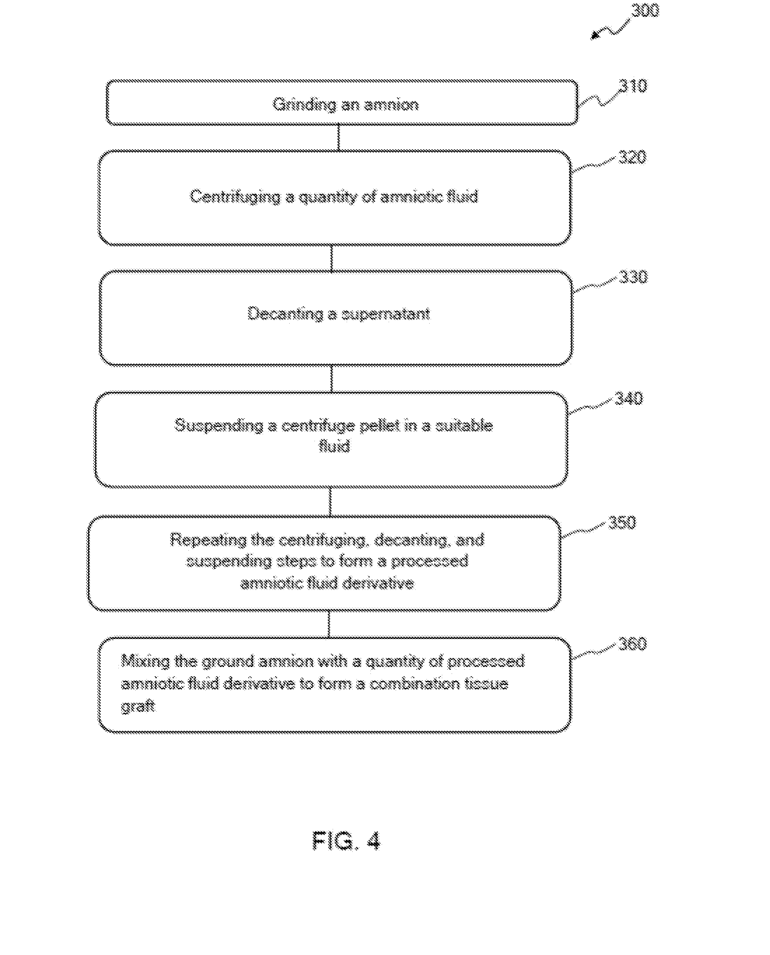

FIG. 4 shows method 300 of forming a reconstituted amniotic membrane-amniotic fluid combination tissue graft 400. Step 310 of method 300 is grinding an amnion. Step 310 is the same as step 210 of method 200 disclosed herein above. Step 320 of method 300 comprises centrifuging a quantity of amniotic fluid. Step 330 of method 300 comprises decanting a supernatant. Step 340 of method 300 comprises suspending a centrifuge pellet in a suitable fluid. Some embodiments of centrifuging step 320, decanting step 330, and suspending step 340 have been fully disclosed in detail herein above and comprise the amniotic fluid washing steps. Step 350 of method 300 comprises repeating the centrifuging, decanting, and suspending steps to form a processed amniotic fluid derivative. In some example embodiments disclosed herein above, centrifuging step 320, decanting step 330, and suspending step 340 are repeated twice following the initial steps for a total of three series of washing steps. This is not meant to be limiting. In some embodiments, method 300 comprises two, three, four, or greater than four series of washing steps. Step 360 of method 400 comprises mixing the ground amnion with a quantity of processed amniotic fluid derivative to form a combination tissue graft 400 similar to step 220 of method 200 disclosed herein above.

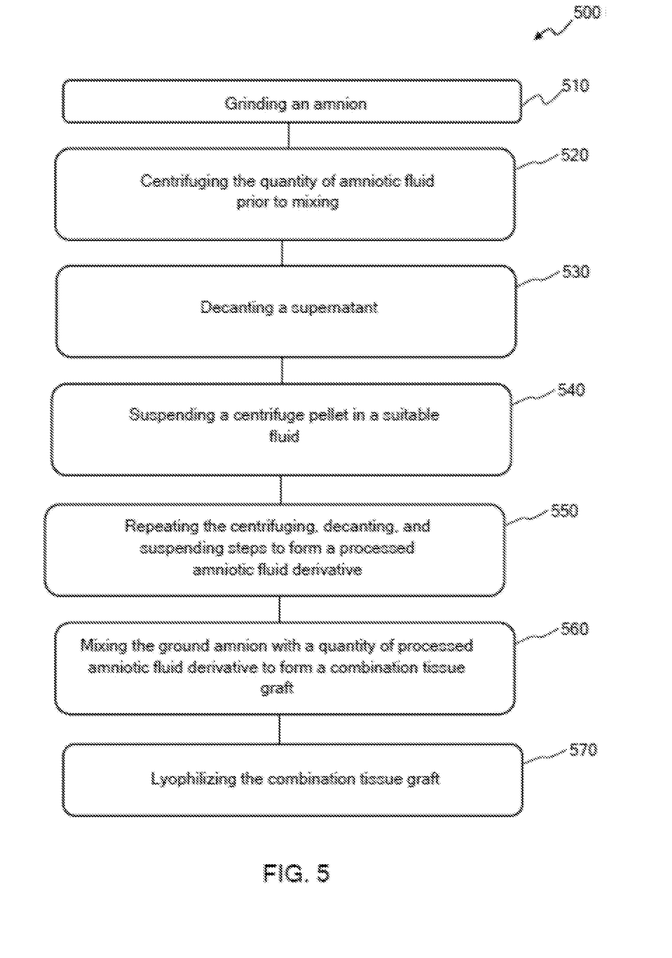

FIG. 5 shows method 500 of forming a combination tissue graft. As shown in FIG. 5, some embodiments comprise grinding step 510, centrifuging step 520, decanting step 530, suspending step 540, repeating step 550, and mixing step 560 similar to steps 310, 320, 330, 340, 350, and 360 of method 300 disclosed herein above. Method 500 further comprises the additional step of lyophilizing the combination tissue graft 400. Lyophilization is performed in one of many possible lyophilization units and protocols known in the art for the purpose of preserving SC viability under long-term storage conditions of frozen combination tissue graft 400.

The embodiments and examples set forth herein were presented in order to best explain the present invention and its practical application, and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above, and are intended to fall within the scope of the appended claims.

What is claimed is:

1. A reconstituted combination tissue graft comprising:
a dried amniotic membrane; and
an amniotic fluid, wherein the amniotic fluid rehydrates the dried amniotic membrane.

2. The combination tissue graft of claim 1, wherein the dried amniotic membrane is morcellized.

3. The combination tissue graft of claim 1, wherein the dried amniotic membrane is ground.

4. The combination tissue graft of claim 1, further comprising a non-amniotic fluid liquid.

5. The combination tissue graft of claim 4, wherein the non-amniotic fluid liquid is an isotonic electrolyte solution.

6. The combination tissue graft of claim 4, wherein the non-amniotic fluid liquid is a cryoprotectant.

7. The combination tissue graft of claim 4, wherein the non-amniotic fluid liquid comprises an isotonic electrolyte solution and a cryoprotectant.

8. The combination tissue graft of claim 1, wherein the amniotic membrane and amniotic fluid are mammalian.

9. The combination tissue graft of claim 1, wherein the amniotic membrane and amniotic fluid are from one individual donor.

10. The combination tissue graft of claim 1, wherein the amniotic membrane and amniotic fluid are from more than one individual donor.

11. The combination tissue graft of claim 1 wherein the combination tissue graft is lyophilized.

12. The combination tissue graft of claim 1 wherein the combination tissue graft is a fluid.

13. The combination tissue graft of claim 1 wherein the combination tissue graft is a semi-solid gel.

14. A reconstituted combination tissue graft comprising:
a dried amniotic membrane;
a processed amniotic fluid derivative comprising a protein released from a disrupted cell; and
a hydrating fluid, wherein the hydrating fluid rehydrates the dried amniotic membrane.

15. The reconstituted combination tissue graft of claim 14, wherein the protein is a growth factor.

16. The combination tissue graft of claim 14, wherein the disrupted cell is an amniocyte.

17. The combination tissue graft of claim 14, wherein the hydrating fluid is an amniotic fluid.

18. A method of forming a combination tissue graft comprising the steps of:
grinding an amnion; and
mixing the ground amnion with a quantity of processed amniotic fluid derivative to form a combination tissue graft.

19. The method of claim 18, further comprising:
centrifuging the quantity of amniotic fluid prior to mixing;
decanting a supernatant;
suspending a centrifuge pellet in a suitable fluid; and
repeating the centrifuging, decanting, and suspending steps to form a processed amniotic fluid derivative.

20. The method of claim 18, further comprising a step lyophilizing the combination tissue graft.
